(19) United States
(12) Patent Application Publication
(10) Pub. No.: US 2012/0077747 A1
(43) Pub. Date: Mar. 29, 2012

(54) FISH-DERIVED PROTEIN LYSATE, AND USES THEREOF AS IMMUNOMODULATORY AND/OR ANTI-INFLAMMATORY AGENT

(75) Inventors: Raphael Anguenot, Rimouski (CA); Martin Beaulieu, Rimouski (CA); Jocelyn Bérubé, Ancienne-Lorette (CA); Sandrine Briate, St-Anaclet (CA)

(73) Assignee: Innovactiv Inc., Rimouski, QC (CA)

(21) Appl. No.: 13/125,185

(22) PCT Filed: Oct. 23, 2009
(86) PCT No.: PCT/CA09/01498

§ 371 Date: Dec. 16, 2011

Related U.S. Application Data
(60) Provisional application No. 61/107,854, filed on Oct. 23, 2008.

(51) Int. Cl. A61K 38/17; A61P 37/00; C12P 21/06; A61P 25/00; A61P 1/12; A61P 1/14; A61P 29/00; A61P 1/00

(52) U.S. Cl. 514/12.2; 514/21.92; 514/18.3; 435/68.1

(57) ABSTRACT

A peptide-comprising extract derived from fish is described. Also disclosed is a process for obtaining a peptide-comprising extract derived from fish, as well as an extract obtained by this process. Compositions comprising such an extract are also described. Uses of such extracts/compositions, as well as corresponding methods of treatment, for example to prevent and/or treat an inflammatory and/or immune disease-related discomfort in a subject, are also described.

### CROSS REFERENCE TO RELATED APPLICATIONS

This application claims priority on U.S. provisional application Ser. No. 61/107,854, filed on Oct. 23, 2008. All documents above are incorporated herein in their entirety by reference.

### STATEMENT REGARDING FEDERALLY SPONSORED RESEARCH OR DEVELOPMENT

N/A.

### FIELD OF THE INVENTION

The present invention relates to the prevention and/or treatment of inflammatory diseases, and more particularly to fish-derived composition and method therefore or uses thereof for the prevention and treatment of inflammatory diseases.

### BACKGROUND OF THE INVENTION

Inflammation is usually a local protective reaction that serves to protect the organism against infection and trauma. Excessive inflammation or unusual activity of an otherwise normal inflammatory process leads to inflammatory diseases.

Inflammation processes are multifactorial. These factors include cellular (endothelial cells, macrophages, leucocytes, etc) and humoral (coagulation factors, cytokines, eicosanoid acids, adhesion molecules, free radicals, etc). In general, inflammatory processes are associated with immune cell proliferation, infiltration of cells and accumulation of exudates at the inflammation site, and degradation of the injured tissue. Liberation of mediators like cytokines and eicosanoid acids leads to a more global reaction like fever. At the end of the inflammation process, the injured tissue is destroyed by the organism.

Exposure of tissue or cells to harmful stimuli such as lipopolysaccharides (LPS), toxic chemicals, radiations or inflammatory mediators are known to activate immune cells to produce inflammatory mediators such as tumor necrosis factor-α (TNF-α), interleukin-1 (IL-1), IL-6, prostaglandins (PG), leukotrienes and nitric oxide (NO).

Prostaglandins are molecules originating from arachidonic acid and involved in various physiological activities and pathologies. Their production is regulated by the enzyme cyclooxygenase (COX). Non-steroidal anti-inflammatory drugs (NSAIDs) and analgesic agents such as aspirin and indomethacin, which inhibit prostaglandins synthesis by inhibiting COX activity, are known to provide an excellent therapeutic effect for inflammation-related diseases.

The activation of nuclear factor κB (NF-κB) is involved in the production of NO, prostaglandins, and TNF-α. NF-κB is also known to regulate the expression of a variety of proteins involved in the various cellular responses such as apoptosis, immune responses, and inflammatory reactions.

Abnormal inflammation processes may induce persistent diseases. Many diseases have an important inflammatory component, amongst which chronic inflammatory diseases are recognized to be polysystemic diseases with unknown origin. They are as diversified as psoriasis, Crohn's disease, ulcerative colitis, rheumatoid arthritis. In general, an immune component can be seen in all of these diseases.

There is a need for an effective treatment of inflammation and autoimmune diseases. Such ailments are generally treated with drugs that have high side effects profiles and limited efficacy. Currently, the most commonly used therapeutic agents available to treat chronic inflammations are corticosteroids or NSAIDs. All of these anti-inflammatory agents have significant side effects, such as gastrointestinal irritation and bleeding, bone loss, and fluid retention, some of which can reduce significantly the well-being of the patients.

There is thus a need for novel composition and methods for the prevention and/or treatment of inflammatory diseases.

The present description refers to a number of documents, the content of which is herein incorporated by reference in their entirety.

### SUMMARY OF THE INVENTION

In the studies described herein, it is demonstrated that oral administration of a peptide-comprising extract obtained from a fish protein isolate modulates the expression of various molecules involved in the inflammation and immune regulation in animal models and in human.

Accordingly, one aspect of the present invention contemplates a method for preventing and/or reducing the effects of an inflammatory and/or immune disease-related discomfort in a subject, the method comprising administering to a subject an effective amount of a fish-derived peptide extract capable of preventing or treating the inflammatory and/or immune disease-related discomfort, as described herein.

In another aspect, the present invention provides a method for preventing and/or treating an inflammatory and/or immune disease-related discomfort comprising administering to a subject in need thereof an effective amount of a peptide-comprising extract derived from a fish organ or tissue.

In another aspect, the present invention provides a peptide-comprising extract derived from a fish organ or tissue for preventing and/or treating an inflammatory and/or immune disease-related discomfort.

In another aspect, the present invention provides a use of a peptide-comprising extract derived from a fish organ or tissue for preventing and/or treating an inflammatory and/or immune disease-related discomfort in a subject.

In another aspect, the present invention provides a use of a peptide-comprising extract derived from a fish organ or tissue for the preparation of a medicament for preventing and/or treating an inflammatory and/or immune disease-related discomfort in a subject.

In another aspect, the present invention provides a method for reducing a symptom and/or secondary effect of a disorder of the digestive tract (e.g., a disorder associated with a disequilibrium of the inflammatory and/or immune response in the digestive tract), the method comprising administering to a subject in need thereof an effective amount of a peptide-comprising extract derived from a fish organ or tissue.

In another aspect, the present invention provides a use a peptide-comprising extract derived from a fish organ or tissue for reducing a symptom and/or secondary effect a disorder of the digestive tract (e.g., a disorder associated with a disequilibrium of the inflammatory and/or immune response in the digestive tract) in a subject.

In another aspect, the present invention provides a use a peptide-comprising extract derived from a fish organ or tissue for the preparation of a medicament for reducing a symptom and/or secondary effect a disorder of the digestive tract (e.g., a disorder associated with a disequilibrium of the inflammatory and/or immune response in the digestive tract) in a subject.

In another aspect, the present invention provides a process for obtaining a peptide-comprising extract derived from a fish organ or tissue, the process comprising contacting a protein-comprising fish organ or tissue with at least one protease under conditions suitable for protease activity, thereby obtaining a peptide-comprising extract.

In another aspect, the present invention provides a peptide-comprising extract obtained by the above-mentioned process.

The term “peptide-comprising extract derived from a fish organ or tissue” generally refers to an extract which comprises one or more fragment(s) of protein(s) that are normally present in a fish organ or tissue. The peptide(s) comprised in the extract may be obtained using methods well known in the art, for example, by chemical synthesis (i.e. synthesis of a fragment of a protein known to be present in a fish organ or tissue), or by fragmentation of protein(s) present in a fish organ or tissue (through enzymatic digestion using one or more protease(s), for example).

The starting material (i.e. a protein-comprising fish tissue or organ, or an extract thereof) to obtain the above-mentioned peptide-comprising extract may be ground/crushed or otherwise broken or sheared into smaller pieces using any method (or device) known in the art, for example using commercially-available grinders or comparable devices. The starting material may freeze-dried before being ground/crushed or otherwise broken up. The ground/crushed material may be lyophilized (e.g., to obtain a powder) and/or stored under proper conditions. The ground/crushed starting material may be submitted to one or more purification steps such as precipitation(s) (e.g., “salting out”), chromatography (e.g., size exclusion chromatography), filtration (e.g., ultrafiltration), which may be useful to remove non-protein material such as sugars (e.g., polysaccharides), lipids, as well as connective tissue material such as collagen. In a specific embodiment, the protein isolate is substantially free (i.e. 70-80%) of lipids and sugars including complex sugars.

Any tissue or organ, or any combinations thereof, obtained from any fish may be used as starting material to obtain the peptide-comprising extract of the present invention (e.g., flesh, skin, gonads, digestive tract, viscera, muscles, respiratory tract). For example, the starting material may be collected from the processing wastes of the industry, for example the food or cosmetic industry.

In an embodiment, the protein-comprising fish tissue or organ extract/isolate (i.e., protein isolate) is enriched at about 70% or more (i.e. protein content of 70% or more), in a further embodiment at about 80% or more.

In an embodiment, the above-mentioned fish is a shark or a herring. In a further embodiment, the above-mentioned shark is School shark (Galeorhinus galeus), Rig shark (Mustelus lenticulatus), Blue shark (Pionance glauca), Black shark (Dahlias licha), or any combination thereof.

In an embodiment, the peptide-comprising extract is obtained by contacting a tissue or organ extract/sample (i.e. tissue or organ extract/sample comprising proteins) with a protease (e.g., a proteolytic enzyme). Any protease (or combination of proteases) which permits to generate peptides from a protein-comprising extract may be used. In an embodiment, the above-mentioned protease is trypsin and/or chemotrypsin.

In an embodiment, the peptide-comprising extract is filtrated on a filter having a pre-determined molecular weight cut-off (e.g., 5 kDa, 10 kDa, 20 kDa, 50 kDa, 100 kDa) to obtain an extract composed of molecules having a molecular weight above or below a certain threshold. In an embodiment, the peptide-comprising extract is passed on a filter having a cut-off of 10 kDa, to obtain an extract composed of molecules having a molecular weight of about 10 kDa or less. This filtration step may be useful, for example, to eliminate undigested proteins, including the protease(s) used for digestion, from the peptide-comprising extract.

In another aspect, the present invention provides a composition comprising the above-mentioned extract and a carrier.

The peptide-comprising extract according to the invention can be mixed with customary pharmaceutically tolerable carriers, diluents or vehicles and, if appropriate, with other auxiliaries molecules, and administered, for examples, topically, orally, parenterally or colorectally. This formulation can be administered orally in the form of granules, capsules, softgel, pills, tablets, film-coated tablets, sugar-coated tablets, syrups, emulsions, suspensions (e.g., ready-to-drink powder), dispersions, aerosols, solutions, and/or liquids. In a specific embodiment it is a capsule or a tablet. In a specific embodiment, the peptide-comprising extract is spray-dried or lyophilized. In an embodiment, the extract/composition of the present invention is formulated in the form of a capsule. It can also be administered as suppositories, vaginal suppositories, and/or parenterally, e.g. in the form of solutions, emulsions, creams or suspensions. It can administer in preparation for time delayed release, or protected from gastric acid by coating to be released in the intestinal part of the gut. Preparations to be administered orally can contain one or more additives such as sweeteners, aromatizing agents, colorants and preservatives. Tablets can contain the composition mixed with customary pharmaceutically tolerable auxiliaries, as for example inert diluents such as calcium carbonate, sodium carbonate, lactose and talc; granulating agents and agents which promote the disintegration of the tablets on oral administration, such as starch or alginic acid; binding agents such as starch or gelatin; and lubricants such as magnesium stearate, strearic acid and talc.

The formulations are prepared, for example, by extending the composition with solvents and/or excipients if appropriate, using emulsifiers and/or dispersants, it being possible, for example, in the case of the use of water as a diluent optionally to use organic solvents as auxiliary solvents.

Administration is carried out in a customary manner, preferably orally or parenterally, or prelingually, sublingually, colorectally, topically or intravenously. In the case of oral administration, apart from the excipients mentioned, tablets can also contain additives, such as sodium citrate, calcium carbonate and dicalcium phosphate together with other various additives, such as starch, preferably potato starch, gelatin and the like. Furthermore, lubricants such as magnesium stearate, sodium lauryl sulphate and talc can additionally be used for tableting. In the case of aqueous suspensions and/or elixirs, which are intended for oral administration, the active compounds can be mixed, apart from with the above-mentioned auxiliaries, with various flavors enhancers or colorants. In the case of parenteral administration, solutions of the composition using suitable liquid excipients can be employed.

Capsules can contain the composition as a single constituent or mixed with a solid diluent such as calcium carbonate, calcium phosphate or kaolin. The injectable formulations are also formulated in a manner known per se.

Furthermore, antioxidants can be added to the composition and to the pharmaceutical and topical formulations. The use of natural or naturally identical compounds such as, for example, tocopherols. The antioxidants contained in the compositions according to the invention, for example, in amounts from 0.01-5% by weight, in particular from 0.5-2% by weight, based on the total composition.

The composition according to the invention can be formulated as liquid, pasty or solid preparations, for example as aqueous or alcoholic solutions, aqueous suspensions or emulsions.

In an embodiment, the composition is formulated to target an inflamed tissue and/or organ, such as the intestine.

In another aspect, the extract and/or compositions comprising the extract of the present invention can be formulated for administration as foods or dietary supplements using one or more consumable carriers. A “consumable carrier” is herein defined as any food, food ingredient, or food additive, or any excipient utilized for tabletting, encapsulation, or other formulation of an active agent for oral administration, whether for human or animal use. For dietary supplements, the extract can be mixed according to methods routine in the art. Dietary supplements can be prepared in a variety of forms including, but not limited to, liquid, powder (e.g., ready-to-drink powder), or solid pill forms. The extract or composition of the present invention can be administered either alone or in combination with other compounds or extracts where combining compounds or extracts would lead to additive or synergistic effects. The extract and/or composition of the present invention can also be added directly to foods and ingested as part of a normal meal. Various methods are known to those skilled in the art for addition or incorporation of such agents into foods.

The term “inflammation” as used herein is intended to represent the normal response of the immune system to infection or irritation.

The terms “inflammatory disease” or “inflammatory disorder” as used herein are intended to mean any disease or disorder affecting the normal response of the immune system to infection or irritation. The disease or disorder can involve either an excessive or an insufficient response on the immune system to infection or irritation, a lack of response following infection or irritation, and a response in the absence of infection or irritation. Such diseases or disorders can be for example, without limitation, ulcerative proctitis, proctosigmoiditis, left-sided colitis, pan-ulcerative (total) colitis, psoriatic arthritis, ankylosing spondylitis, juvenile ankylosing spondylitis, seronegative enthesopathy, arthropathy syndrome, Reiter's syndrome, spondyloarthropathies, and/or endogenous uveitis.

In an embodiment, the inflammatory disease is a chronic inflammatory disease. Chronic inflammation disease includes rheumatoid arthritis, psoriasis, cutaneous inflammation, atopic dermatitis, encephalitis, hypersensitivity pneumonitis, chronic lung inflammation, ischemia-reperfusion injury, systemic lupus erythematosus, myositis, ankylosing spondylitis, sceroderma, acute inflammatory demyelinting polyradiculoneuropathy, vasculitis, appendicitis, arachnoiditis, myocarditis, acute cholecystitis, chronic airflow obstruction, chronic hepatitis, chronic obstructive pulmonary disease, conjunctivitis, dermatitis, enteritis, gingivitis, hepatitis, ileitis, asthma, and/or chronic inflammation diseases of the digestive tract such as Crohn's disease, irritable bowel syndrome, inflammatory bowel disease or ulcerative colitis.

The term “inflamed tissue” as used herein refers to a tissue affected by inflammation and affected cells contained within the tissue. Preferably, the inflamed tissue is from a mammalian species, preferably a human.

The expressions “effective amount” and “therapeutically amount” as used herein are intended to mean an amount sufficient to initiate a beneficial or desired clinical result, such as an improvement of the condition of the patient. An effective amount can be administered in one or more doses. For the purposes of this invention, an effective amount on the present composition is an amount that induces a therapeutic or prophylactic response against at least one factor responsible for inflammation. Such amount will vary according to the nature of the inflamed tissue, the severity of the inflammation, the mode of administration, etc. One skilled in the art can easily and without difficulty monitor the inflamed tissue so as to determine what will be such effective amount. In an embodiment, the effective amount is about 100 mg to about 1000 mg per day, in a further embodiment about 200 mg to about 800 mg per day, in a further embodiment about 300 mg to about 700 mg per day, in a further embodiment about 400 mg to about 600 mg per day, in a further embodiment about 500 mg per day. In a preferred embodiment, the extract and composition of the present invention may be taken once a day at a dose of about 300 mg.

In an embodiment, the above-mentioned extract, composition, method, or use, induces the production of one or more anti-inflammatory and/or immunoregulatory mediators in the subject. In a further embodiment, the above-mentioned anti-inflammatory and/or immunoregulatory mediator is IL-6, IgA, IL-10, IL-4, CTLA-4, transforming growth factor-beta (TGF-β), or any combination thereof, in the subject.

In an embodiment, the above-mentioned discomfort, symptom or secondary effect is abdominal pain, abdominal cramp, diarrhea, flatulence (gas), bloating, constipation, irregularity, or any combination thereof. In an embodiment, the above-mentioned discomfort, symptom or secondary effect is associated with an inflammatory and/or immune-related disorder of the digestive tract, such as an infection, infectious diarrhea, irritable bowel syndrome, inflammatory bowel disease.

The extract or composition of the present invention may further comprise at least one of further agent known to be useful for the prevention and/or treatment of an inflammatory disease, or may be used in combination with another agent known to be useful for the prevention and/or treatment of an inflammatory disease (e.g., combination therapy).

The term individual, subject or patient according to this invention is intended to mean a vertebrate, preferably a mammal, more preferably a human. The extract/composition of the present invention may be used in veterinary applications, i.e. for the treatment of inflammatory-related disease/disorder in mammals including, but not limited to, farm animals, sport animals, rodents, primates, and pets.

### BRIEF DESCRIPTION OF THE DRAWINGS

In the appended drawings:

FIG. 1 shows IL-6 production by intestine epithelial cells (IECs) ex vivo culture (isolated from untreated mice) after addition of 1) LPS (positive control, 0.1 μg/ml); 2) 6.25 μg/ml of a shark protein hydrolysate (TH); 3) TH 6.25 μg/ml+TLR2 (culture in presence of anti-TLR2 antibodies); 4) TH 6.25 μg/ml+TLR4 (culture in presence of anti-TLR4 antibodies); 5) water (negative control); 6) Unhydrolyzed fish proteins negative control) (6 μg/ml); 7) Unhydrolyzed fish proteins (control) (6.25 μg/ml)+TLR2 (culture in presence of anti-TLR2 antibodies); and 8) Unhydrolyzed fish proteins (control)(6.25 μg/ml)+TLR4 (culture in presence of anti-TLR4 antibodies). Data presented as mean±SD.

FIG. 2 shows the effect of oral administration of total hydrolysates (TH) for 2 and 7 days on the number of IgA+ cells; * indicates statistically significant difference as compared to the control (p<0.05);

FIG. 3 shows the effect of oral administration of the TH for 2 and 7 days on the number of IgG* cells;

FIG. 4 shows the effect of oral administration of the TH (4.5 mg/kg of mice bw) (equivalent to 300 mg in human) for 7 days on serum IgA antibody titers in mice. Data presented as mean±SD*p<0.05 Vs baseline;

FIG. 5 shows the effect of oral administration of the TH for 2 and 7 days on the number of (A) IL-6+, (B) IL-10+, (C) IL-4+, (D) IFN-γ+, (E) TNF-α+ and (F) CTLA-4+ cells in the lamina propria and on (G) the TGF-β level in the serum. * indicates statistically significant difference as compared to the control (p<0.05);

FIG. 6 shows a size-exclusion elution profile at 280 nm of a protein lysate. Peptides were dissolved at 4 mg/ml in 0.1 M phosphate buffer (pH 6.8). (A) Sample (10 μl) was loaded on a BIOSEP-SEC-S2000™ (Phenomenex) gel filtration column at a flow rate of 1 ml/min using an elution buffer consisting of 0.1 M phosphate buffer (pH 6.8). (B) a mix of molecular weight standards was resolved under the same conditions; and

FIG. 7 shows a SDS-PAGE profile of a protein lysate. Peptides were solubilized at 300 mg/ml in deionized water and dialysis overnight against water. 300 μg (lane 1), 600 μg (lane 2) and 1200 μg (lane 3) of dialysed peptides were resolved on a 15% SDS-PAGE gel using standard running conditions. Molecular weights in kDa are indicated at the left. BSA=Serum bovine albumine, TRP=porcine trypsine; and

FIG. 8 shows the effect of oral administration of the TH (300 mg/day) for 28 days on serum IgA antibody titers in healthy male volunteers. Data presented as mean±SD.

### DESCRIPTION OF ILLUSTRATIVE EMBODIMENTS

The present invention is illustrated in further details by the following non-limiting examples.

### Example 1

### Preparation of Fish Protein Hydrosylate

Protein extracts from shark flesh (obtained from Waitaki Biosciences, New Zealand, Cat. No. 930480) were used as raw material for the studies described herein. A typical amino acid profile of the protein extracts is provided in Table I. Marine hydrolysates (MH) were produced using enzymatic digestion by trypsin from porcine pancreas (Sigma-Aldrich, Cat. No. 10303; CAS number 9002-07-7) and type II a-Chymotrypsin from bovine pancreas (Sigma-Aldrich, Cat. No. C4129; CAS number 9004-07-3), using a concentration of 2.5% to 5% (w/vol) of protein extract in water. The hydrolysis reaction was performed at pH 8.0 and 35° C. to 45° C. The pH-stat technique (Adler-Nissen, 1977) was used to maintain the enzyme preparation at its optimal pH. The enzymatic hydrolysate was ultrafiltered using a 10-kDa cut-off membrane in order to remove the protease and the non-hydrolyzed proteins. The retentate or reaction mixture was discarded whereas permeate, so-called total hydrolysate (TH) (example of peptide-containing extract), was further characterized and used for evaluation of their functional properties.

TABLE I
Typical amino acid profile of a shark protein extract

| AMINO ACIDS | mg/g | % w/w |
|---|---|---|
| Alanine | 55.9 | 6.3 |
| Arginine | 62.1 | 7.0 |
| Aspartic Acid | 90.3 | 10.1 |
| Cystine | 10.2 | 1.2 |
| Glutamic Acid | 138.0 | 15.5 |
| Glycine | 53.7 | 6.1 |
| Histidine | 21.9 | 2.5 |
| Isoleucine | 37.7 | 4.3 |
| Leucine | 71.7 | 8.1 |
| Lysine | 77.9 | 8.8 |
| Methionine | 26.6 | 3 |
| Phenylalanine | 35.7 | 4 |
| Proline | 37.4 | 4.2 |
| Serine | 40.8 | 4.6 |
| Threonine | 46.8 | 5.3 |
| Trytophan | 10.7 | 1.2 |
| Tyrosine | 30.4 | 3.4 |
| Valine | 39.0 | 4.4 |
| | 886.8 | 100.0 |

### Example 2

IL-6 Production by Intestine Epithelial Cells (IECs) Ex Vivo Culture

### Animals and Feeding Procedures:

6 to 8 weeks-old BALB/c female mice weighing 20 to 25 g were obtained from Charles River (Montreal, Canada). Each experimental group (sample) consisted in 5 mice housed together in plastic cages kept in a controlled atmosphere (temperature: 22±2° C.; humidity: 55±2%) with a 12 h light/dark cycle. Mice were maintained and treated in accordance with the guidelines of the Canadian Council on Animal Care.

Animals were offered an aqueous solution of protein hydrolysate (TH) (0.30 mg/ml or 100 μg) for 2 or 7 consecutive days (in replacement of water). All groups of mice received simultaneously a conventional balanced diet ad libitum. A control group received the same conventional balanced diet, but with water instead of the TH. At the end of each feeding period, animals were anesthetized and sacrificed by cervical dislocation to obtain the different tissues for the immunological studies.

### Primary Culture of Mouse Small- and Large-Intestine Epithelial Cells (IEC):

Preparation of primary cultures of enterocytes was performed at the end of each period of the oral administration of protein hydrolysate solution (ex vivo assays) or for the in vitro assays, animals were anesthetized and sacrificed by cervical dislocation. The small and large intestines were removed and placed in Hanks' balanced salt solution (HBSS) (Sigma-Aldrich, St. Louis, Mo.) containing glucose (2%; Sigma-Aldrich), penicillin (100 U/ml; Sigma-Aldrich), and streptomycin (0.1 mg/ml; Sigma-Aldrich). The intestines were flushed six times with 10 ml of the same buffer, cut into 2- to 3-mm fragments, and collected in HBSS. The large intestine was treated with 5 mM dithiothreitol (Sigma) for 15 min at 37° C. to remove the mucus. Then, both the small and large intestines were digested in 20 ml of HBSS containing collagenase (300 U/ml; Sigma-Aldrich C-7657) and dispase (0.1 mg/ml; Gibco, Grand Island, N.Y.) at 25° C. and 150-rpm agitation for 45 min and 60 min, respectively. Digestion was stopped by the addition of 20 ml of Dulbecco's modified Eagle medium (DMEM) without phenol red (Gibco) supplemented with heat-inactivated fetal bovine serum (10%; ATCC, Manassas, Va.), epidermal growth factor (10 ng/ml; U.S. Biological, Swampscott, Mass.), insulin-transferrin-selenium-A (2.50 μg/ml, 0.55 μg/ml, and 1.68 pg/ml, respectively) from a 100× ready-to-use solution (Gibco), penicillin (100 U/ml; Sigma-Aldrich), and streptomycin (0.1 mg/ml; Sigma-Aldrich). Large fragments were removed using gravity by allowing them to settle (2 min) at the bottom of the flask. The supernatant was transferred to centrifuge tubes and centrifuged for 3 min at 300 rpm. The pellet was washed twice with the culture medium and finally resuspended in the same culture medium at a concentration of 4×10⁵ to 6×10⁵ organoids (single cells or IEC cluster)/ml. IEC suspensions were then transferred to 96-well cell culture plates (200 μl/well) and incubated for 8 h (37° C.; 5% CO₂). An in vitro toxicology assay kit, 3-(4,5-dimethylthiazol-2-yl)-2,5-diphenyltetrazolium bromide (MTT) based (Sigma-Aldrich), and trypan blue (0.4%) exclusion were used to assess cell viability. Supernatants were recovered for cytokine determination. IL-6 was determined using the corresponding mouse IL-6 enzyme-linked immunosorbent assay set (BD OptEIA™; BD Biosciences Pharmingen, San Diego, Calif.).

Blocking of IEC with Antibodies Against Toll-Like Receptor 2 (TLR2) and Toll-Like Receptor 4 (TLR4), and Challenge with Bacterial Strains:

IEC suspensions from untreated mice were obtained as described above and incubated for 30 min at room temperature in the presence of 40 μg/ml (10 times the blocking concentration suggested by the manufacturer) of functional grade purified anti-mouse TLR4/MD2, anti-mouse TLR2 (eBioscience, San Diego, Calif.), or Dulbecco's modified Eagle medium (control). After the blocking period with anti-TLRs, the functional dose (6 μg/ml) of the TH was added. LPS (lipopolysaccharides, a bacterial endotoxin) was used as a positive control at a final concentration of 0.1 μg/ml. The IEC suspensions were incubated for 8 h (37° C.; 5% CO₂), and supernatants were recovered and kept frozen until IL-6 quantification by ELISA.

### Statistical Analysis:

Data were analyzed using the one-way analysis of variance procedure of SPSS software. The differences among means were detected by Duncan's multiple-range test (SPSS software). Data were considered significantly different when the p value was <0.05.

### Results:

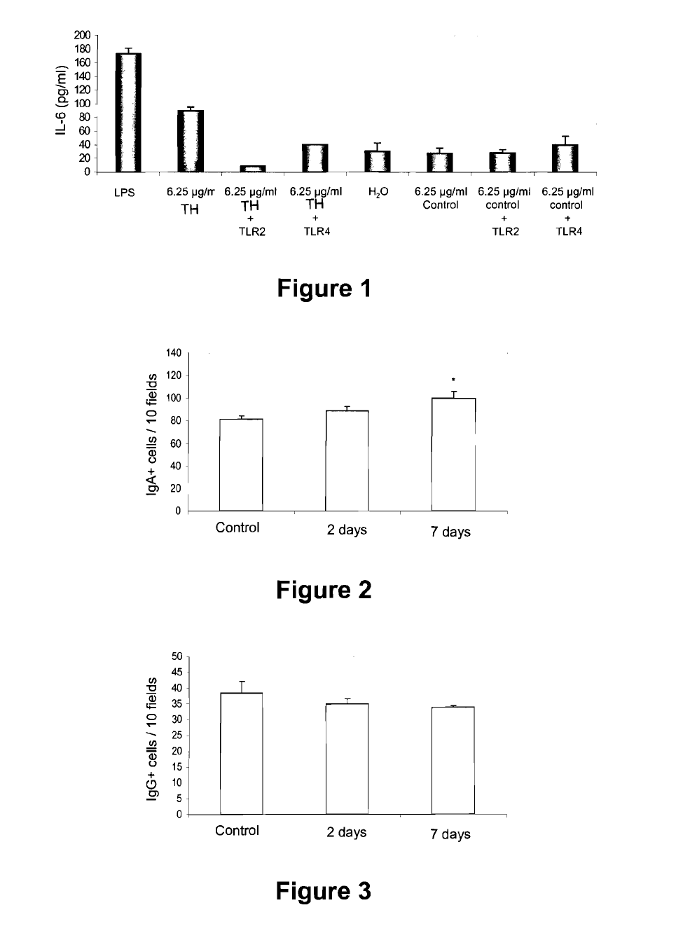

The ex vivo experiment by culturing Intestinal Epithelial Cells (IECs) with different concentrations of TH sought to determine the functional dose, and to compare the production of IL-6 by IECₛ to LPS stimuli. The experiment also sought to determine the effect of blocking TLR-2 and 4 (i.e. probiotic assumed mechanism of action) on IL-6 production by IECs in the presence of the TH and the negative control (i.e. unhydrolysed fish proteins of Example 1; were shown herein not to possess tested activity) (FIG. 1).

The positive control LPS (bacterial endotoxin) generated a strong immune reaction as measured on Interleukin-6 (IL-6) release as expected since soluble toxins such as LPS typically provoke antibody-mediated immune responses through IL-6. Native proteins (i.e. unhydrolysed proteins of Example 1) have no immunomodulatory effect.

The TH generated an intermediary reaction, half way between the massive response generated by LPS and that generated by the negative control (water). This level of stimulation seems sufficient to trigger an increase in B-lymphocyte differentiation and proliferation into antibody-producing plasmocytes, but without reaching levels at which it could become pro-inflammatory. (Duarte et al., 2006 Immunobiology 211: 341-350).

As can be deduced by comparing the effect the non-hydrolyzed fish proteins with that of the TH, the hydrolysis process generated immuno-modulatory peptides able to stimulate IL-6 release, whereas native proteins have no effect.

When TLR-2 and 4 were blocked by their respective antibodies, the production of IL-6 by IECs exposed to TH decreased, as shown in FIG. 1. Without being bound by any particular theory, this suggests that the immune cascade is triggered at least in part by the binding to TLRs. Binding or partial binding to both TLRs might be involved in better protection against enteric infections by competition with pathogens for adhesion sites. Moreover, TLR-4 is involved in the activation of NF-κB, which plays a role in the inflammatory reaction.

### Example 3

Effect of Oral Administration of TH to Mice on B Cell Populations (IgA⁺ and IgG⁺ B Cells) and Circulating Secreted IgA

### Animals and Feeding Procedures:

6 to 8 weeks-old BALB/c female mice weighing 20 to 25 g were obtained from Charles River (Montreal, Canada). Each experimental group (sample) consisted in 5 mice housed together in plastic cages kept in a controlled atmosphere (temperature 22±2° C.; humidity 55±2%) with a 12 h light/dark cycle. Mice were maintained and treated in accordance with the guidelines of the Canadian Council on Animal Care.

Animals were offered the TH (0.30 mg/ml or 100 μg) (corresponds to 4.5 mg/kg of mice body weight) for 2 or 7 consecutive days (in replacement of water). All groups of mice received simultaneously a conventional balanced diet ad libitum. A control group received the same conventional balanced diet, but with water instead of the TH. At the end of each feeding period, animals were anesthetized and sacrificed by cervical dislocation to obtain the different tissues for the immunological studies.

### Protocol:

Small intestines were removed for histological preparation following Sainte-Marie technique (1962) for paraffin inclusion. The number of IgA-producing (IgA⁺) cells was determined on histological slices of samples from the ileum region near Peyer's patches by a direct immunofluorescence method. Blood samples were also obtained and circulating IgA measured with fluorescent monoclonal antibodies.

The number of IgG-producing (IgG⁺) cells was also determined on histological slices of small intestine. The immuno-fluorescence test was performed using (α-chain specific) anti-mouse IgA FITC conjugate or (γ-chain specific) anti-mouse IgG FITC conjugate (Sigma-Aldrich, St. Louis, Mo., U.S.A.). Histological slices were deparaffinized and rehydrated in a graded series of ethanol. Deparaffinized histological samples were incubated with the appropriate antibody dilution ( 1/100 for IgA or 1/50 for IgG) in PBS solution for 30 min at 37° C. Then, samples were washed 2 times with PBS solution and examined using a fluorescent light microscope. The results were expressed as the number of IgA⁺ or IgG⁺ cells (positive: fluorescent cell) per 10 fields (magnification 100×). Data represent the mean of three histological slices for each animal, for each feeding period.

### Results:

The number of IgA⁺ cells increased in the lamina propria of mice fed with TH for 2 and 7 days (FIG. 2). Humoral immunity mediated by secretory IgA antibodies is involved in the defense against mucosal infections. Thus, the biological effect of enhanced IgA⁺ B-cells is mostly related to increased immune surveillance to prevent intestinal infections and other pathologies. IgA is also correlated to the maintenance of intestinal homeostasis, and may play a role as a barrier against neoplasia in the mucosal territories (Wiseman et al., 2007 Can J Surg 50:3; 223).

The level of IgG⁺ cells (FIG. 3) was maintained at normal levels in mice fed with the TH.

Increased IgG would normally be correlated with increased permeability of the intestinal barrier which occurs as a sign of inflammation. Here, no structural damage was detected at any level of the intestines, as confirmed by histological analysis. In addition, translocation studies at the liver showed negative Enterobacter culture. Hence, the absence of IgG stimulation observed in this experiment, coupled with negative bacterial translocation to the liver, is indicative of an overall improved mucosal barrier.

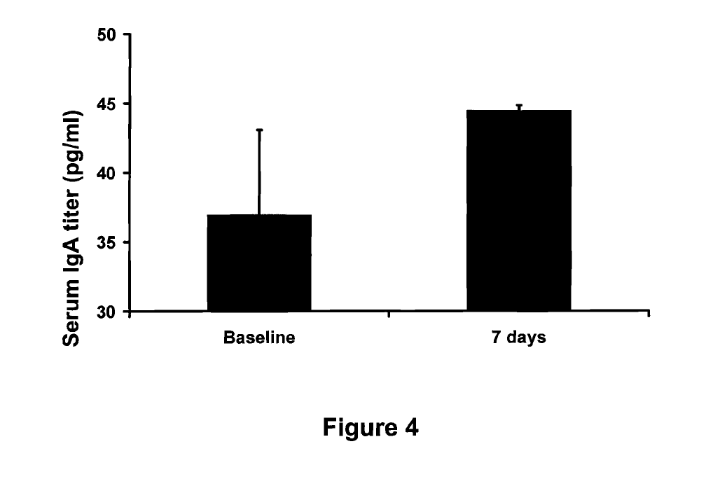

Serum IgA titers have been determined and compared between baseline and after 7 days of treatment with the TH (see FIG. 4). At such a low dose and short duration of administration, mice saw their mucosal immunity improved as demonstrated by an augmentation in local and systemic IgA secretion.

### Example 4

Effect of Oral Administration of Th to Mice on Expression of Immunoregulatory/Inflammatory Mediators

### Animals and Feeding Procedures:

6 to 8 weeks-old BALB/c female mice weighing 20 to 25 g were obtained from Charles River (Montreal, Canada). Each experimental group (sample) consisted in 5 mice housed together in plastic cages kept in a controlled atmosphere (temperature 22±2° C.; humidity 55±2%) with a 12 h light/dark cycle. Mice were maintained and treated in accordance with the guidelines of the Canadian Council on Animal Care.

Animals were offered a TH (0.30 mg/ml or 100 μg) for 2 or 7 consecutive days (in replacement of water). All groups of mice received simultaneously a conventional balanced diet ad libitum. A control group received the same conventional balanced diet, but with water instead of the TH. At the end of each feeding period, animals were anesthetized and sacrificed by cervical dislocation to obtain the different tissues for the immunological studies.

### Protocol:

To determine the role of the TH on the recruitment of TH1 and TH2 lymphocytes, the number of IL-4+, IL-6+, IFN-γ+ and TNF-α+ cells in the small intestine lamina propria and the serum concentration of TGF-β were determined. At the end of each feeding period, the small intestine was thus removed and processed for histological preparation as described above. IL-4, IL-6, IL-10, TNF-α, CTLA-4 and IFN-γ expressions were studied by an indirect immunofluorescence method. Histological slices were deparaffinized and rehydrated in a graded series of ethanol, and then incubated for 30 min in a 1% blocking solution of BSA (Jackson Immuno Research, West Grove, Pa., USA) at room temperature. Histological slices were then incubated for 60 min at 37° C. with rabbit anti-mouse IL-4, IL-6, IL-10, TNF-α or IFN-γ (Peprotech, Inc., Rocky Hill, N.J., U.S.A) polyclonal antibodies. The incubation was followed by two washes with PBS solution and, finally, sections were treated for 45 min at 37° C. with a dilution of a goat anti-rabbit antibody conjugated with FITC (Jackson Immuno Research). Data represent the mean of three histological slices for each animal, for each feeding period. TGF-β was analyzed in the serum by ELISA.

### Results:

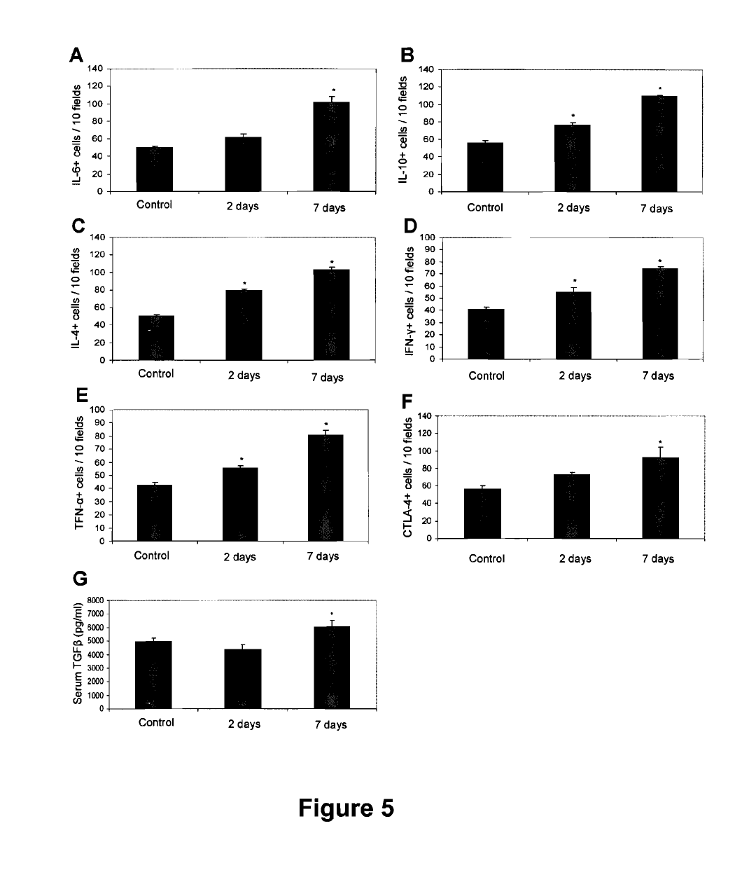

To determine the effect of oral administration of the TH on the expression of inflammatory/immunoregulatory mediators, the number of IL-4⁺, IL-6⁺, IL-10⁺, IFN-γ⁺, TNF-α⁺, CTLA-4⁺ cells in the small intestine lamina propria and TGF-β in the serum were determined (FIGS. 5A to 5G). IL-4⁺, IL-6⁺ and IL-10⁺ cells were all significantly increased in the lamina propria of the intestine in mice fed with the TH, which is in accordance with the increased levels of IgA+ cells observed in Example 3 above. TGF-β was increased in the serum. TH2-promoting cytokines IL-6+ and IL-4+ were thus significantly increased in the lamina propria of the intestine. TH1-promoting cytokines INFα and IFNγ were also significantly increased by approximately the same order of magnitude, hence maintaining a healthy balance between TH1 and TH2-types of immune responses. Without being bound to any particular theory, the increase in TNF-α and IFN-γ levels, which is immune-controlled by IL-10, might be associated with the priming of the immune response by increasing epithelial stimulation and initiating the cross-talk between the associated immune cells. The increased expression of IL-10⁺ cells and TGF-β in combination supports the anti-inflammatory effects of the TH and its tissue reparative potential.

Upregulation of CTLA-4 protein expression (FIG. 5F) is typically associated with downregulation of overall T-cell function, including proliferation, and thus with immunoregulatory function.

Inflammation was thus maintained in a healthy stage as demonstrated by negative liver bacterial translocation and increased regulatory T cells function demonstrated by increased IL-10, CTLA-4 and TGF-β1 synthesis. These elements help improve the physical barrier of the intestinal mucosa (by reducing inflammation) as well as passive immune protection associated with IgA secretion. The TH also increased the recruitment of TH1 and TH2 lymphocytes by upregulating cytokines IL4 and IL-6 (TH2-promoting cytokines), as well as IFNγ and TNF-α (TH1-promoting cytokines).

### Example 5

### Characterization of the Shark-Derived Protein Lysate

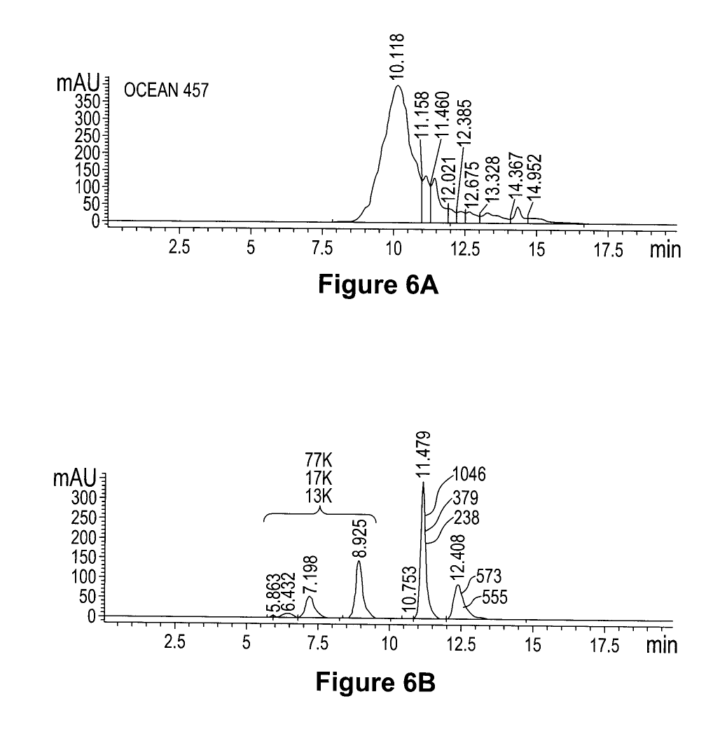

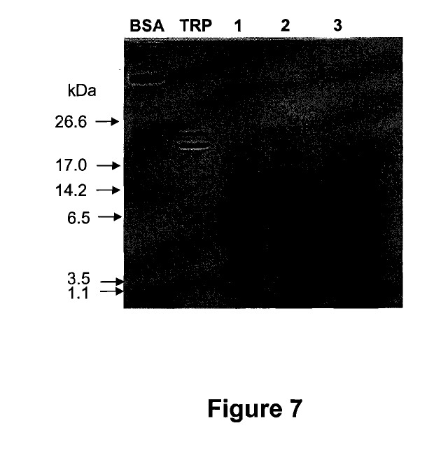

The peptide elution profile (size-exclusion chromatography) and the SDS-PAGE profile of the TH is shown at FIGS. 6 and 7, respectively.

The spray-dried TH is free flowing, very soluble in water and may be compressed.

Marine taste and odour of the TH compared to that of other commercially available products and raw material was slight.

In accordance with specific e TH is compliant for heavy metals with requirements of California's Prop 65. There is no other contaminant listed likely to arise from the raw material or from the manufacturing processing of the TH. The TH complies with the most stringent standards for allowable limits of contaminant in food, including those of the Canadian Food Inspection Agency (CFIA), the U.S. Environmental Protection Agency (EPA) and California's Proposition 65 (Prop 65).

### Example 6

### Preclinical In Vivo Safety Studies

A state-of-the-art animal safety study (standard GLP protocol) was performed in an independent GLP laboratory in mice and rats. This study showed that the TH administered at a daily dose of 430 mg/kg (100 times higher than the recommended daily dose in human) during 30 consecutive days is well tolerated at that treatment level. Absence of toxicity and inflammation was shown by organ weight and negative liver translocation. Hepatic enzymes, blood chemistry and behavior remained normal during the study.

### Example 7

### Effect of Oral Administration of the TH to Human on IgA Antibody Titer

The TH has been studied in a randomized, double-blinded, placebo-controlled clinical trial in order to evaluate its potential for modulating the immune response of healthy subjects. This trial was conducted in 47 healthy men and women aged 20 to 60. After giving their written consent, participants were randomized to receive either 300 mg of the TH or a placebo on a daily basis, for 28 consecutive days. This trial was conducted in an independent, University-affiliated research organization. Table 1 below presents baseline characteristics of participants. Baseline characteristics and compliance were similar in both groups. Side effects occurred at similar frequencies in both treatment arm.

TABLE 1
Characteristics of subjects¹ in each treatment group at baseline,
analysis of side effects during the study and compliance.

| | TH | Placebo |
|---|---|---|
| | (n = 23) | (n = 24) |
| Gender | Women: 13 | Women: 12 |
| | Men: 10 | Men: 12 |
| Age (yr) | 42.9 ± 12.4 | 40.2 ± 14.7 |
| BMI (kg/m2) | 25.1 ± 2.6 | 25.4 ± 3.3 |
| DBP (mm Hg) | 69.9 ± 5.6 | 70.5 ± 6.7 |
| SBP (mm Hg) | 106.6 ± 9.1 | 108.0 ± 11.1 |
| Side effects | | |
| After day 14 | 69.6% | 62.5% |
| After day 28 | 65.2% | 66.7% |

| | |
|---|---|
| Compliance | 99.4% |

¹Subjects who completed the study

Participants were subjected to blood and saliva sampling at day 0, and after 14 and 28 days of the TH or the placebo intake. Those samples were used for titration of total serum and salivary IgA content.

The primary endpoint in this trial was a change in serum IgA levels.

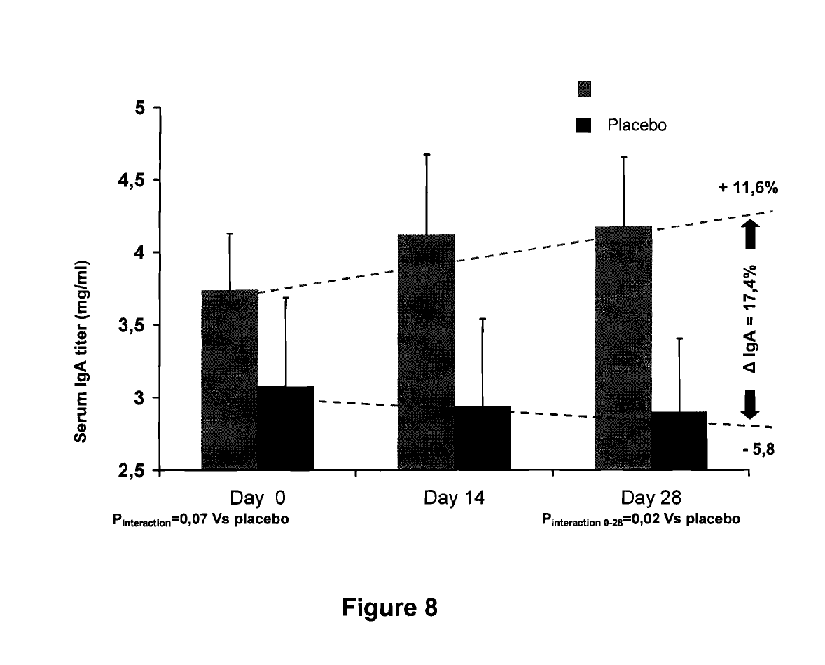

As shown in FIG. 8, the TH induced an increase in circulatory IgA levels in healthy male volunteers. This increase was seen at day 14 and was maintained at day 28. The increase of 17.4% over placebo is similar in magnitude to what was obtained in animal trials (20% as shown in FIG. 4). Statistical analyses were conducted by ANOVA and were normalized for baseline value difference.

Initial analysis of the entire cohort was hindered by highly variable results obtained in the women subgroup. Serum IgA levels are known to be influenced by age, mood, and gender. Women generally have lower IgA levels than men, and also have varying IgA levels throughout their hormonal cycle (higher in the follicular phase than in the luteal phase) (Gomez E, Ortiz V, Saint-Martin B, Boeck L, Diaz-Sanchez V, Bourges H. Hormonal regulation of the secretory IgA (sIgA) system: estradiol- and progesterone-induced changes in sIgA in parotid saliva along the menstrual cycle. Am J Reprod Immunol. 1993 May; 29(4):219-23). Lack of standardization for the hormonal effect in the women subgroup may explain the high variability obtained in this population.

Although the present invention has been described hereinabove by way of specific embodiments thereof, it can be modified, without departing from the spirit and nature of the subject invention as defined in the appended claims.

1. A method for preventing and/or treating an inflammatory and/or immune disease-related discomfort comprising administering to a subject in need thereof an effective amount of a peptide-comprising extract derived from a fish organ or tissue.

2. The method of claim 1, wherein the organ or tissue is skin, flesh, viscera, a gonad, or any combination thereof.

3. The method of claim 2, wherein the organ or tissue is flesh.

4. The method of claim 1, wherein the peptide-comprising extract is obtained by contacting the fish organ or tissue with a protease.

5. The method of claim 4, wherein the protease is trypsin, chemotrypsin, or a combination thereof.

6. The method of claim 1, wherein the peptide-comprising extract is composed of molecules having a molecular weight of about 10 kDa or less.

7. The method of claim 1, wherein the fish is a shark or a herring.

8. The method of claim 7, wherein the shark is School shark (Galeorhinus galeus), Rig shark (Mustelus lenticulatus), Blue shark (Pionance glauca), Black shark (Dalitias licha), or any combination thereof.

9. The method of claim 1, wherein the inflammatory and/or immune disease is an inflammatory and/or immune disease of the digestive tract.

10. The method of claim 9, wherein the inflammatory or immune disease of the digestive tract is infectious diarrhea, irritable bowel syndrome or an inflammatory bowel disease.

11. The method of claim 9, wherein the discomfort is abdominal pain, abdominal cramp, diarrhea, flatulence, bloating, constipation, irregularity, or any combination thereof.

12. The method of claim 1, wherein said method induces the production of interleukin- (IL-) 6, IgA, IL-10, IL-4, Cytotoxic T-Lymphocyte Antigen 4 (CTLA-4), transforming growth factor-beta (TGF-β), or any combination thereof, in the subject.

13. The method of claim 1, wherein the effective amount is about 300 mg per day.

14. The method of claim 1, wherein the extract is administered in the form of a capsule, softgel or ready-to-drink powder.

15.-20. (canceled)

21. The method of claim 1, wherein the extract is in the form of a spray-dried powder.

22.-27. (canceled)

28. A composition comprising the extract as defined in claim 1, and a carrier.

29. The composition of claim 28, wherein the carrier is a pharmaceutically acceptable carrier.

30. The composition of claim 29, wherein the composition is in the form of a capsule, softgel or ready-to-drink powder.

31. The composition of claim 28, wherein the carrier is a consumable acceptable carrier.

32.-44. (canceled)

45. A food product comprising the extract as defined in claim 1.

46. A process for obtaining a peptide-comprising extract derived from a fish organ or tissue, the process comprising contacting a protein-comprising fish organ or tissue with at least one protease under conditions suitable for the protease activity, thereby obtaining a peptide-comprising extract.

47. The process of claim 46, wherein the organ or tissue is skin, flesh, viscera, a gonad, or any combination thereof.

48. The process of claim 47, wherein the organ or tissue is flesh.

49. The process of claim 46, wherein the at least one protease is trypsin, chemotrypsin, or a combination thereof.

50. The process of claim 49, wherein the conditions suitable for protease activity comprises a pH of about 8.0 and a temperature of about 35° C. to about 45° C.

51. The process of claim 46, further comprising filtering the peptide-comprising extract on a filter having a cut-off of about 10 kDa, and collecting the filtrate.

52. The process of claim 46, wherein the fish is a shark or a herring.

53. The process of claim 52, wherein the shark is School shark (Galeorhinus galeus), Rig shark (Mustelus lenticulatus), Blue shark (Pionance glauca), Black shark (Dalitias licha), or any combination thereof.

54. The process of claim 46, wherein the protein-comprising fish organ or tissue is lyophilized prior to the contacting step.

55.-58. (canceled)

59. A peptide-comprising extract obtained by the process of claim 46.
